(19) United States
(12) Patent Application Publication
(10) Pub. No.: US 2025/0101448 A1
(43) Pub. Date: Mar. 27, 2025

(54) Protein, Gene and Their Vectors and Applications that Control Symbiotic Nitrogen Fixation Efficiency of Soybeans

(71) Applicant: Henan University, Kaifeng, Henan (CN)

(72) Inventors: Xuelu WANG, Kaifeng (CN); Xiaolong KE, Kaifeng (CN)

(21) Appl. No.: 18/725,738

(22) PCT Filed: Apr. 25, 2023
(86) PCT No.: PCT/CN2023/090521

§ 371 Date: Jun. 28, 2024

(30) Foreign Application Priority Data
Nov. 19, 2022 (CN) 202211450181.9

(51) Int. Cl. C12N 15/82; C07K 14/415

(52) U.S. Cl. CPC C12N 15/8261; C07K 14/415

(57) ABSTRACT

The present invention belongs to the field of biotechnology, relates to new applications of genes, and in particular to proteins, genes and their editing vectors and applications to control the symbiotic nitrogen fixation efficiency of soybeans. The present application discovers two soybean genes, GmNAS1, GmNAP1, and one transcription factor, GmNFYC10a, and the gene sequences of the three are obtained through reverse genetics. The soybean hairy root transient transformation system, stable transgenic plants, PCR reaction, and gene sequencing are successfully isolated, cloned and verified. The interaction between soybean genes GmNAS1, GmNAP1 and the transcription factor GmNFYC10a controls the symbiotic nitrogen fixation efficiency of soybean, revealing the molecular mechanism by which GmNAS1, GmNAP1 and GmNFYC10a improve the nitrogen fixation efficiency of root nodules by regulating the direction of PEP flow.

### FIELD OF INVENTION

The present invention belongs to the technology field of biotechnology, and is related to a new application of gene, which is in particularly related to proteins, genes and their vectors and applications that control the symbiotic nitrogen fixation efficiency of soybeans.

### DESCRIPTION OF RELATED ARTS

Cultivated soybean [Glycine max (L.) Merr] originated in our country (China) and is an important food, feed, oilseed and energy crop in the world. It is also an important source of protein for humans. Since the mid-1990s, our country has become a net importer of soybeans, and the import volume has increased year by year. By 2019, soybean import volume in our country has reached 88.51 million tons, ranking first in the world. Therefore, development of soybean production in our country and achieving “Soybean revitalization” is of great significance to ensuring national food security.

At the same time, with the development of our country's economy and the pressure on food demand caused by population surge, our country uses a large amount of chemical fertilizers in food production, of which nitrogen fertilizers are the most common type. According to statistics from the World Food and Agriculture Organization (FAO), annual nitrogen fertilizer use in our country has continued to increase from 2002 to 2014. Although cultivated land area in our country only accounts for 8% in the world's, the nitrogen fertilizer use has reached 35% of the world's total use (http://faostat.fao.org/). Excessive application of nitrogen fertilizer and nutrient loss have led to serious eutrophication pollution of water bodies; it has also affected the composition of soil organic matter, reduced biological activity, and led to soil compaction. Nitrogen-fixing microorganisms can use their own synthesized nitrogenase to reduce atmospheric nitrogen into ammonia that can be directly utilized by plants. In the global ecosystem, the nitrogen content fixed by leguminous plants and Rhizobia through symbiotic nitrogen fixation accounts for 60%-70% of the total biological nitrogen fixation. The symbiotic nitrogen fixation between leguminous plants and Rhizobia not only reduces energy consumption, but also helps improve soil, and is an important way to improve the nitrogen efficiency of leguminous crops. In the agricultural system, the nitrogen fixation of soybeans accounts for more than 86% of the total symbiotic nitrogen fixation of beans, and the annual nitrogen production reaches 16.44 million tons. Therefore, improving the efficiency of symbiotic nitrogen fixation of soybeans and Rhizobia is of great significance to the development of green and sustainable agriculture.

Like other leguminous plants, soybeans can form root nodules in symbiosis with Rhizobia, thereby assimilating nitrogen in the atmosphere into ammonia and providing a nitrogen source for themselves. Designing and cultivating soybean varieties with efficient nitrogen fixation will help increase soybean yields while reducing chemical nitrogen fertilizer use, thus ensuring the sustainable development of agriculture. Previous studies have shown that the nitrogen-fixing ability of root nodules of many leguminous plants is highly susceptible to environmental changes. Abiotic stresses such as drought, high salt, and low light can significantly inhibit the nitrogen-fixing ability of root nodules. The increase in carbon dioxide concentration in the atmosphere and the lack of nitrogen sources in the soil can improve the nitrogen fixation ability of root nodules. Therefore, designing and cultivating highly efficient nitrogen-fixing soybean varieties with broad adaptability urgently requires us to conduct more in-depth research on the regulatory mechanism of soybean root nodule nitrogen-fixing ability in response to environmental changes.

As a place for carbon and nitrogen exchange between host leguminous plants and Rhizobia, mature root nodules can receive the sucrose synthesized by photosynthesis of the aboveground parts through the phloem after formation. The sucrose after reaching the root nodule will be decomposed into hexose by sucrose synthase and alkali invertase. Sugar then enters the glycolysis pathway to produce phosphoenolpyruvate (phosphoenolpyruvate, PEP). PEP can generate malic acid through a two-step reaction catalyzed by phosphoenolpyruvate carboxylase (phosphoenolpyruvate carboxylase, PEPC) and malate dehydrogenase (malate dehydrogenase, MDH). Malic acid is transported into the bacteroid for the tricarboxylic acid cycle to produce ATP to provide energy for the nitrogen fixation reaction. The ammonium ions generated by nitrogen fixation are then assimilated into asparagine and glutamine in the amide-exporting legume (amide-exporting legume) and transported to the aboveground parts. Among ureide-exporting leguminous plants (ureide-exporting legume) represented by soybeans, the glutamine generated from the ammonium radical enters the de novo purine biosynthesis (de novo purine biosynthesis) pathway to generate inosine monophosphate (de novo purine biosynthesis, IMP), and finally converted into urea and transported to the aboveground parts. In addition, PEP can also generate pyruvate, the final product of glycolysis, under the catalysis of pyruvate kinase (pyruvate kinase, PK), and then the pyruvate enters the tricarboxylic acid cycle of mitochondria to produce ATP to provide energy for nitrogen assimilation in root nodules.

### SUMMARY OF THE PRESENT INVENTION

### Technical Problems

Although the carbon and nitrogen metabolism pathways of soybean root nodules have been relatively clearly studied, the genes and regulatory mechanisms that regulate these metabolic pathways to improve the nitrogen fixation efficiency of soybean root nodules have not yet been elucidated. How to regulate the nitrogen fixation efficiency of soybean root nodules and how to utilize this regulation Mechanism to improve the nitrogen fixation capacity of soybeans and thereby improve the utilization rate of nitrogen elements in soybeans is a technical problem to be solved by this research group.

### Technical Solutions

In order to solve the above problems in the existing technology, the present invention provides a protein, a gene and its editing vector and application that control the symbiotic nitrogen fixation efficiency of soybeans.

The technical solution of the present invention is realized as follows:

A protein that controls the nitrogen fixation efficiency of soybean symbiosis, the protein has an amino-terminal chloroplast transfer peptide, four CBS functional domains, a PB1 functional domain and a carboxyl-terminal transmembrane domain.

Furthermore, the protein is localized in a mitochondrion and interacts with a transcription factor GmNFYC10a.

Preferably, the protein is localized to the mitochondrion through a transmembrane domain at a carboxyl terminus.

In the above proteins that control the symbiotic nitrogen fixation efficiency of soybeans, the proteins are GmNAS1 protein and GmNAP1 protein, wherein an amino acid sequence of the GmNAS1 protein is shown in SEQ ID NO.4, an amino acid sequence of the GmNAP1 protein is shown in SEQ ID NO.5, and an amino acid sequence of the transcription factor GmNFYC10a is shown in SEQ ID NO.6.

The nucleotide sequence of the GmNAS1 gene encoding the GmNAS1 protein is shown in SEQ ID NO.1, the nucleotide sequence of the GmNAP1 gene encoding the GmNAP1 protein is shown in SEQ ID NO.2, and the nucleotide sequence encoding the transcription factor GmNFYC10a is shown in shown in SEQ ID NO.1.

An overexpression vector containing the above-mentioned GmNAS1 gene and/or GmNAP1 gene.

An application of the above-mentioned proteins or genes is improving plant symbiotic nitrogen fixation efficiency, enhancing plant environmental adaptability, and increasing plant yield.

An application of the above-mentioned overexpression vector is improving plant symbiotic nitrogen fixation efficiency, enhancing plant environmental adaptability, and increasing plant yield.

Furthermore, the step is: transferring the overexpression vector described in claim 6 through a genetic transformation method into the plant subject to be improved, and then screening and culturing the plant which is transgenic positive to complete the application of enhancing the symbiotic nitrogen-fixing ability of soybeans.

Preferably, the plant is a leguminous plant.

### Beneficial Effect

1. The present application discovers two soybean genes, GmNAS1 and GmNAP1, and one transcription factor GmNFYC10a, and obtains the gene sequences of the three through reverse genetics. By utilizing soybean hairy root transient transformation system, stabilizing transgenic plants, and carrying out PCR reaction, the gene sequencing is isolated, cloned and verified successfully. The interaction between the soybean genes GmNAS1, GmNAP1 and the transcription factor GmNFYC10a controls the efficiency of symbiotic nitrogen fixation in soybean. When the nodule carbon source supply increases, GmNAS1 can sense the decrease in AMP content caused by the increase in nodule energy status, thus promoting more GmNAS1 and GmNAP1 proteins to forms a homodimer. The homodimer of GmNAS1 and GmNAP1 binds to the transcription factor GmNFYC10a on the mitochondria, reducing the nuclear content of GmNFYC10a, thereby reducing the expression of the pyruvate kinase gene and reducing the conversion of PEP to pyruvate, causing more PEP to form oxaloacetate and malic acid, increasing the carbon source supply of bacteroids in the root nodules, and thus enhancing the nitrogen fixation ability of the root nodules. This study reveals that GmNAS1, GmNAP1 and GmNFYC10a regulate the PEP flow direction to improve the molecular mechanism of nitrogen fixation efficiency in root nodules.

2. The two soybean genes GmNAS1, GmNAP1 and one transcription factor GmNFYC10a in this application controls the increase in nitrogen fixation efficiency of root nodules when the carbon source supply of soybeans increases. By overexpressing related genes in soybeans, it will further enhance the operation of related signaling pathways and increase the carbon source supply of bacteroids in root nodules, thus further enhancing the nitrogen fixation efficiency of soybean root nodules.

3. The soybean family homologous genes GmNAS1 and GmNAP1GmNAS1 and GmNAP1 of a pair of soybean cystathionine R synthase (Cystathionine Psynthase, CBS) overexpress in soybean will further improve the nitrogen-fixing ability of root nodules when the carbon source supply is sufficient. Specifically, reducing the nuclear accumulation of a transcription factor GmNFYC10a to redistributes the flow direction of carbon sources in root nodules, thereby enhancing the nitrogen fixation ability of soybean root nodules and hence increasing the above-ground biomass of the plant and the final yield. The inventor's research will provide genetic resources for nitrogen efficiency and molecular breeding of leguminous plants, including soybeans, and provide a theoretical basis for the molecular design of efficient nitrogen-fixing soybean varieties, thereby promoting the development of environmentally friendly green and sustainable agriculture.

### BRIEF DESCRIPTION OF THE DRAWINGS

FIG. 1 shows the expression patterns of GmNAS1 and GmNAP1 genes in public transcriptome databases.

FIG. 2 shows GUS staining of pGmNAS1:GUS and pGmNAP1:GUS transgenic hairy root nodules.

FIG. 3 shows the protein functional domains of GmNAS1 and GmNAP1.

FIG. 4 shows the localization of cTP-GFP-GmNAS1 and GmNAP1-GFP-TMR fusion proteins in tobacco epidermal cells.

FIG. 5 shows the localization of cTP-GFP-GmNAS1 and GmNAP1-GFP-TMR fusion proteins in nodule cells.

FIG. 6 shows the Western blot detection of GmNAS1 and GmNAP1 proteins in different cell fractions.

FIG. 7 shows the localization of GmNAS1-CFP and GmNAP1-CFP fusion proteins in tobacco epidermal cells.

FIG. 8 shows the isothermal titration calorimetry method to detect the binding of GmNAS1 to AMP/ADP/ATP/cAMP.

FIG. 9 shows the isothermal titration calorimetry method to detect the binding of GmNAP1 to AMP/ADP/ATP/cAMP.

FIG. 10 shows the isothermal titration calorimetric method to detect the binding of GmNAS1 protein lacking the CBS functional domain to AMP.

FIG. 11 shows the nodulation phenotype analysis of Ri-GmNAS1/NAP1 transgenic hairy roots.

FIG. 12 shows the nodulation phenotype analysis of cr-nas1, cr-nap1, cr-nas1nap1-1 and cr-nas1nap1-2 transgenic plants, where (A) illustrates a schematic representation of gene editing sites of cr-nas1, cr-nap1, cr-nas1nap1-1 and cr-nas1nap1-2 mutant; (B) illustrates the detection of protein content of GmNAS1 and GmNAP1 in cr-nas1, cr-nap1, cr-nas1nap1-1 and cr-nas1nap1-2 mutants; (C-E) illustrates the analysis of the nodulation phenotype of cr-nas1, cr-nap1, cr-nas1nap1-1 and cr-nas1nap1-2 mutants.

FIG. 13 shows the interaction between GmNAS1/GmNAP1 and GmNFYC10a in mitochondria, wherein (A) is the co-immunoprecipitation mass spectrometry detection of GmNAP1; (B) is the interaction between GmNAS1/GmNAP1 and GmNFYC10a by BiFC detection; (C) is the interaction between GmNAS1/GmNAP1 and GmNFYC10a by pull-down detection.

FIG. 14 shows that the energy status of soybean root nodules regulates the nuclear localization of GmNFYC10a, wherein (A) is the fluorescence localization of GmNFYC10a-GFP in tobacco epidermal cells; (B) is the oligomycin or AMP treatment to induce the nuclear import of GmNFYC10a protein in root nodule cells; (C) is the statistical analysis of GmNFYC10a-GFP fluorescence localization.

FIG. 15 shows that GmNAS1/GmNAP1 affects the glycolysis pathway of soybean root nodules, wherein (A-B) is differentially expressed genes in Ri-EV and Ri-GmNAS1/NAP1 transgenic hairy root nodules; (C) is KEGG enrichment analysis of downregulated genes in Ri-GmNAS1/NAP1 root nodules; (D) is glycolysis genes which is downregulated in Ri-GmNAS1/NAP1 nodules.

FIG. 16 shows the glycolysis pathway of soybean root nodules activated by GmNFYC10a, wherein (A) is the regulatory motif analysis of 10 glycolysis gene promoters; (B) is the ChIP-qPCR analysis of GmNFYC10a and PK1a/GAPC1/PK2a promoters; (C) is the transcriptional activation analysis of GmNFYC10a and PK1a/GAPC1/PK2a promoters.

FIG. 17 shows that the distribution of PEP in soybean nodules being regulated by GmNAS1/GmNAP1 and GmNFYC10a, wherein (A) is the detection of GmNFYC10a protein content in the nucleus of nodules before and after sucrose treatment; (B) is the detection of expression levels of glycolysis genes in root nodules before and after sucrose treatment; (C) is the detection of expression levels of glycolytic genes in W82, cr-nas1, cr-nap1and cr-nas1nap1-1 root nodules after sucrose treatment; (D-F) is the contents of pyruvate, oxaloacetate, and malate in W82, cr-nas1, cr-nap1, and cr-nas1nap1-1 root nodules after sucrose treatment, and the content ratio of pyruvate to oxaloacetate.

### THE BEST MODE EMBODIMENT OF THE PRESENT INVENTION

This application requests the protection of a protein that controls the nitrogen fixation efficiency of soybean symbiosis, the protein has an amino-terminal chloroplast transfer peptide, four CBS functional domains, a PB1 functional domain and a carboxyl-terminal transmembrane domain.

Furthermore, the protein is localized in a mitochondrion and interacts with a transcription factor GmNFYC10a.

Preferably, the protein is localized to the mitochondrion through a transmembrane domain at a carboxyl terminus.

In the above proteins that control the symbiotic nitrogen fixation efficiency of soybeans, the proteins are GmNAS1 protein and GmNAP1 protein, wherein an amino acid sequence of the GmNAS1 protein is shown in SEQ ID NO.4, an amino acid sequence of the GmNAP1 protein is shown in SEQ ID NO.5, and an amino acid sequence of the transcription factor GmNFYC10a is shown in SEQ ID NO.6.

The nucleotide sequence of the GmNAS1 gene encoding the GmNAS1 protein is shown in SEQ ID NO.1, the nucleotide sequence of the GmNAP1 gene encoding the GmNAP1 protein is shown in SEQ ID NO.2, and the nucleotide sequence encoding the transcription factor GmNFYC10a is shown in shown in SEQ ID NO.1.

### Detailed Description of the Embodiment of the Present Invention

### Embodiment 1

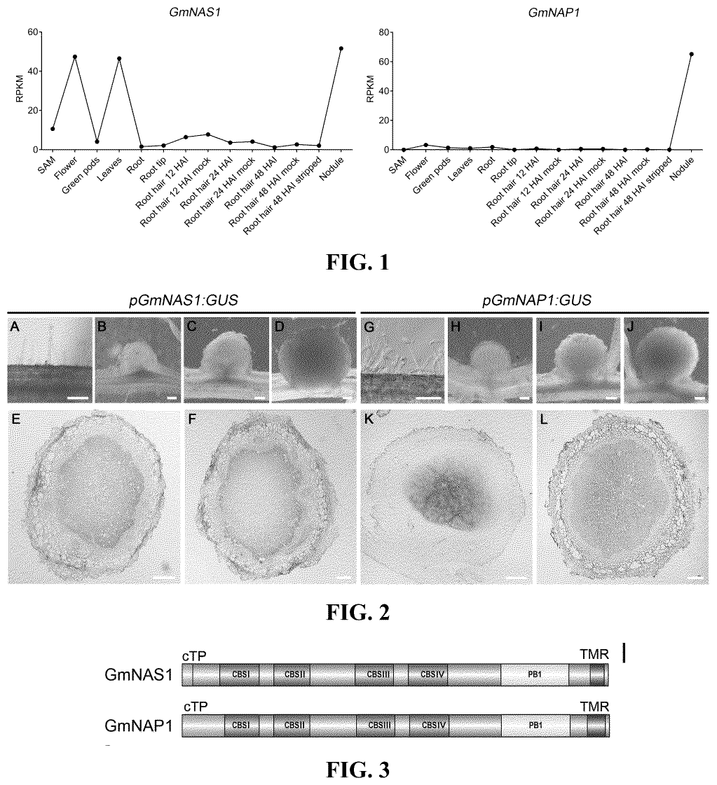

According to this application, search for the expression of soybean CBS family genes in the soybean gene transcriptome database published online, and identify a pair of homologous genes GmNAS1 and GmNAP1 that are highly expressed in soybean root nodules (FIG. 1).

The gene sequence of GmNAS1 is obtained by the following method:

The total volume of the reaction system is 50 μl, the template is 1 μl of soybean line W82 genomic DNA (about 50 ng), 5 μl of 10×KOD enzyme reaction buffer, 2 μl of 25 mM MgCL₂, 5 μl of 5 mM dNTP, and 5 μl of 5 M primers (primers NAS1-F and NAS1-R, each primer is 2.5 μl), 1 μl of KOD enzyme, and ddH₂O (sterile deionized water) being added until 50 μl.

The reaction procedure is: Denature at 94° C. for 5 minutes; 94° C. 30 s; 55° C. 1 min; 68° C. 4 min for 35cycles; extension at 68° C. 10 min.

The primers are:

NAS1-F:
ATGAGCACCACTCAAGCTTC;
NAS1-R:
TTACTGTCTAGAGCGCTTTA.

Finally, a gene sequence containing the nucleotides described in SEQ ID NO.1 is obtained, and the protein encoded by this gene is shown in SEQ ID NO.4.

The protection content of the present invention also includes the nucleotide sequence corresponding to the amino acid sequence shown in SEQ ID NO.4, and also includes proteins having the same function obtained from modifying the sequence as shown in SEQ ID NO.4.

The gene sequence of GmNAP1 is obtained by the following method:

The total volume of the reaction system is 50 μl, the template is 1 μl of soybean line W82 genomic DNA (about 50 ng), 5 μl of 10×KOD enzyme reaction buffer, 2 μl of 25 mM MgCL₂, 5 μl of 5 mM dNTP, and 5 μl of 5 μM primers (primers NAP1-F and NAP1-R, each primer is 2.5 μl), 1 μl of KOD enzyme, and ddH₂O (sterile deionized water) being added until 50 μl.

The reaction procedure is: Denature at 94° C. for 5 minutes; 94° C. 30 s; 55° C. 1 min; 68° C. 3.5 min for 35cycles; extension at 68° C. 10 min.

The primers are:

NAP1-F:
ATGAGCAGCACTGAAACTTC;
NAP1-R:
TTACTGTCTAGAGCGCTTTA.

Finally, a gene sequence containing the nucleotides described in SEQ ID NO.2 is obtained, and the protein encoded by this gene is shown in SEQ ID NO.5.

The protection content of the present invention also includes the nucleotide sequence corresponding to the amino acid sequence shown in SEQ ID NO.5, and also includes proteins having the same function obtained from modifying the sequence as shown in SEQ ID NO.5.

The gene sequence of GmNFYC10a is obtained by the following method:

The total volume of the reaction system is 50 μl, the template is 1 μl of soybean line W82 genomic DNA (about 50 ng), 5 μl of 10×KOD enzyme reaction buffer, 2 μl of 25 mM MgCL₂, 5 μl of 5 mM dNTP, and 5 μl of 5 μM primers (primers NFYC10a-F and NFYC10a-R, each primer is 2.5 μl), 1 μl of KOD enzyme, and ddH₂O (sterile deionized water) being added until 50 μl.

The reaction procedure is: Denature at 94° C. for 5 minutes; 94° C. 30 s; 55° C. 1 min; 68° C. 2 min for 35cycles; extension at 68° C. 10 min.

The primers are:

NFYC10a-F:
ATGGCTTCCTCCAACACTCC;
NFYC10a-R:
TTAACTTCCTCCTTTTCCTG.

Finally, a gene sequence containing the nucleotides described in SEQ ID NO.3 is obtained, and the protein encoded by this gene is shown in SEQ ID NO.6.

The protection content of the present invention also includes the nucleotide sequence corresponding to the amino acid sequence shown in SEQ ID NO.6, and also includes proteins having the same function obtained from modifying the sequence as shown in SEQ ID NO.6.

### Embodiment 2

By constructing GmNAS1 and GmNAP1 gene promoters (the nucleotide sequences of the promoters are shown in SEQ ID NO. 7 and SEQ ID NO. 8 respectively), driving vectors pGmNAS1:GUS and pGmNAS1:GUS of reporter GUS gene expression, transforming soybean hairy roots, and carrying out GUS staining on positive roots and nodules obtained. It is found that GmNAS1 and GmNAP1 are not expressed in the early stages of root and nodule development, but GmNAS1 is expressed in all areas of mature nodules, and GmNAP1 is only highly expressed in infected area of mature nodules (FIG. 2).

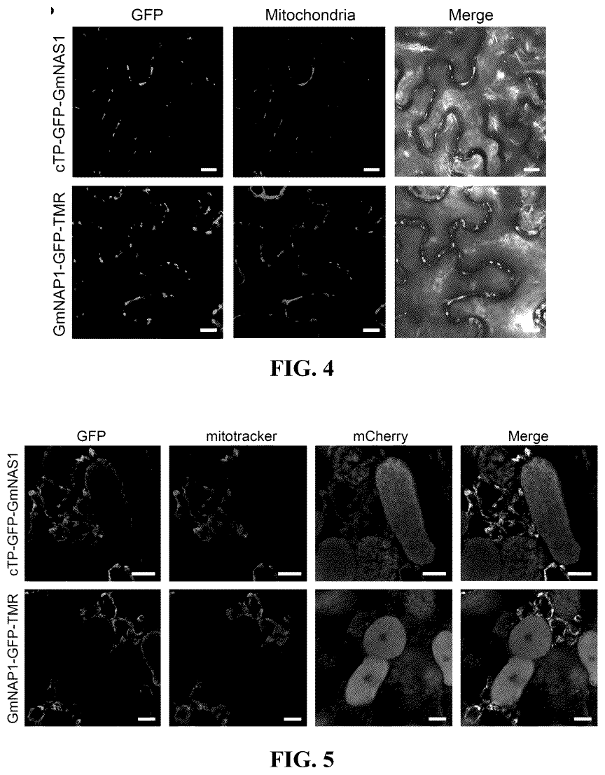

The GmNAS1 and GmNAP1 proteins are expected to have an amino-terminal chloroplast transit peptide (chloroplast transit peptide, cTP), four CBS functional domains, one PB1 (Phox and Bem1) functional domain and one transmembrane region at the carboxyl-terminal (transmembrane region, TMR) (FIG. 3).

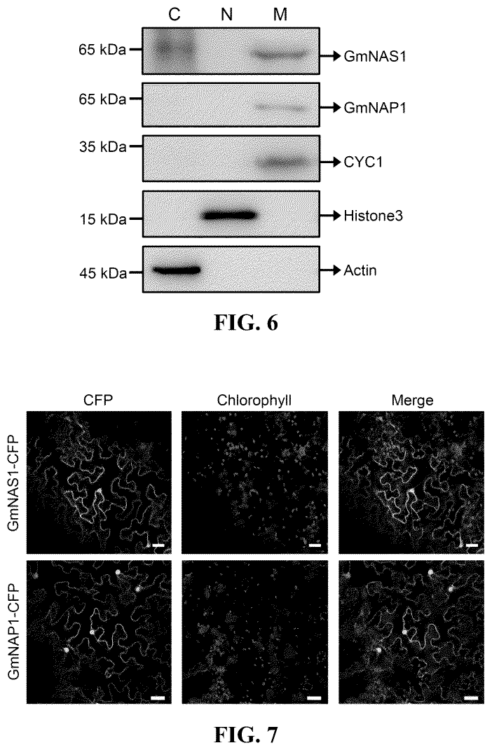

Construct vectors p35S:cTP-GFP-GmNAS1 and p35S: GmNAP1-GFP-TMR and transform Agrobacterium tumefaciens GV3101; inject the successfully transformed Agrobacterium tumefaciens GV3101 into tobacco leaves, and utilize laser confocal microscopy to observe GFP fluorescence in leaf epidermal cells. It is found that the fluorescence are localized in mitochondria (FIG. 4). Construct vectors pGmNAS1:cTP-GFP-GmNAS1 and pGmNAP1:GmNAP1-GFP-TMR and transform Agrobacterium rhizogenes K599; utilize the successfully transformed Agrobacterium rhizogenes K599 to infect soybean root callus, obtain soybean hairy roots and inoculate soybean hairy roots with Bradyrhizobia USDA110, after 25 days, slice the nodules on the hairy roots and observe the GFP fluorescence in the cells in the nodule slices using a laser confocal microscope. It is found that the fluorescence are localized in mitochondria (FIG. 5). Harvest the soybean root nodules, utilize density gradient centrifugation technology to separate the mitochondria, cytoplasm and nuclear components of the nodule cells, then extract the proteins of each component, and detect the protein amounts of GmNAS1 and GmNAP1. It is found that GmNAS1 and GmNAP1 are only detected in mitochondria components (FIG. 6). Construct vectors p35S:GmNAS1-CFP andp35S:GmNAP1-CFP and transform Agrobacterium tumefaciens GV3101; inject the successfully transformed Agrobacterium tumefaciens GV3101 into tobacco leaves, and utilize laser confocal microscopy to observe CFP fluorescence in leaf epidermal cells. It is found that the fluorescence are localized in in the nucleus and cytoplasm, indicating that the mitochondrial localization of GmNAS1 and GmNAP1 requires the help of the transmembrane domain at the carboxyl terminus (FIG. 7).

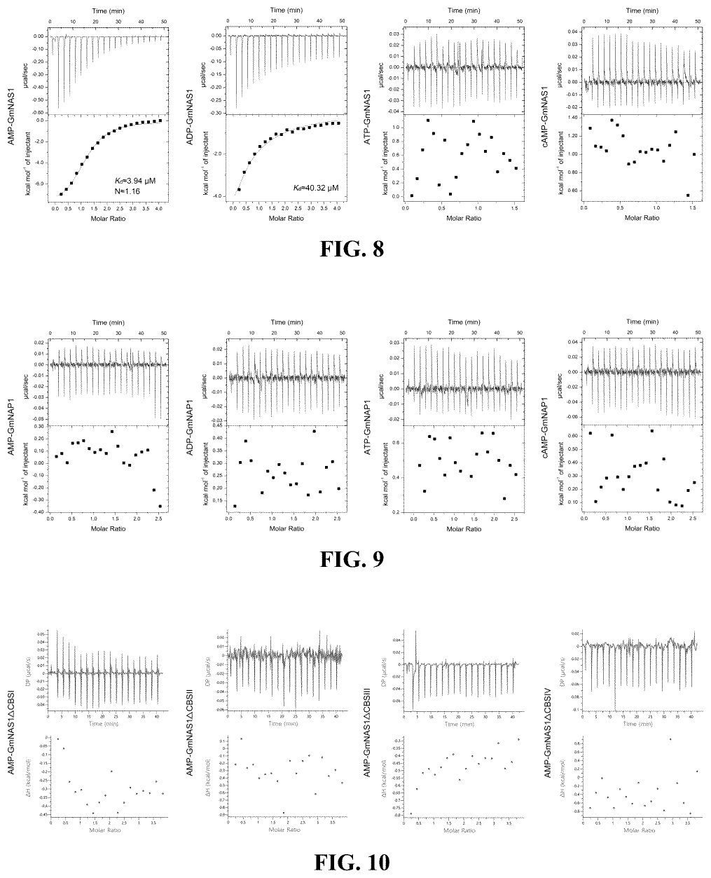

Utilize the prokaryotic expression system of Escherichia coli to express and purify GmNAS1 and GmNAP1 proteins; and then utilize isothermal titration calorimetry to detect the binding ability of GmNAS1/GmNAP1 to AMP, ADP, ATP and cAMP. It is found that GmNAS1 is capable of binding to AMP and ADP, and the dissociation constants are 3.94 M and 40.32 M respectively; and GmNAS1 does not bind to ATP and cAMP (FIG. 8). GmNAP1 does not bind to AMP, ADP, ATP and cAMP (FIG. 9), and the GmNAS1 protein lacking any one CBS functional domain is not capable of binding to AMP (FIG. 10).

### Embodiment 3

Construction and application of GmNAS1 and GmNAP1 gene interference vectors:

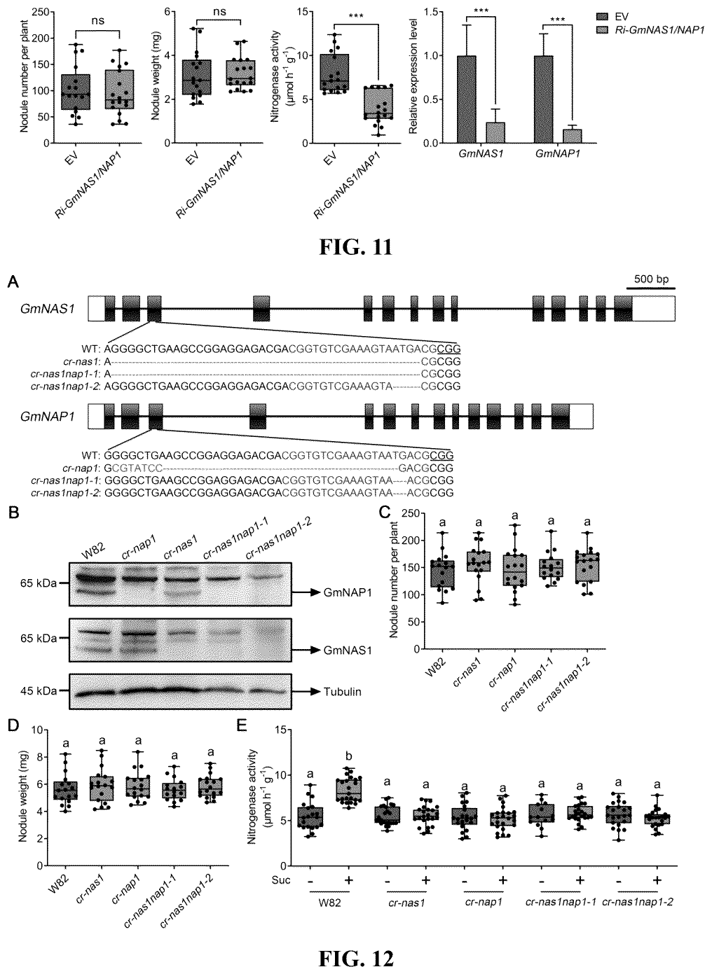

The GmNAS1 and GmNAP1 genes in the soybean line W82 are subjected to RNAi in the hairy root transformation system. After the obtained transgenic hairy roots are inoculated with USDA110, phenotypic statistics of the formed root nodules are performed on the 25th day. It is found that both the number and weight of the root nodules of the Ri-GmNAS1 and GmNAP1 transgenic hairy roots remain unchanged but the activity of nitrogenase of the root nodules is decreased significantly (FIG. 11).

### (1) Construction of Plant Expression Vector Ri-GmNAS1/NAP1

Amplify a 411 bp fragment with high homology in the coding regions of GmNAS1 and GmNAP1 genes from W82; utilize AscI/SwaI and AvrII/BamHI restriction sites for restriction digestion, and connect to the RNAi vector pG2RNAi2 to construct the Ri-GmNAS1/NAP1 recombinant vector.

The primers are as follows:

Ri-GmNAS1/NAP1-F:
ACTCCTAGGGGCGCGCCCAAGCTTCCTCCAACAACAAG
Ri-GmNAS1/NAP1-R:
ACTGGATCCATTTAAATCACAACAGGGAGGTGTCTAAATC

(2) Transformation of Soybean Hairy Roots Mediated by Agrobacterium rhizogenes K599

### 1) Sterilization and Germination Soybean Seed

(a) Seed Sterilization. Pick the soybeans without damage and lesions and sterilize them with chlorine gas (measure 100 ml of sodium hypochlorite and place it in a 250 ml beaker, slowly add 5 ml of concentrated hydrochloric acid along the wall of the beaker, then seal and sterilize for 16 hours).

(b) Seed Germination. Sow sterilized soybean seeds in sterilized quartz sand and place them in a soybean artificial climate chamber for 4 to 5 days to grow until the cotyledons are erect and approximately at right angles to the hypocotyl.

2) Preparation of Agrobacterium rhizogenes with Target Recombinant Vector

Introduce the recombinant plasmid into Agrobacterium rhizogenes K599 to obtain engineered bacteria; activate the engineered bacteria in a liquid culture medium, take 500 μl of the engineered bacteria liquid and spread on a plate, and culture at 28° C. for about 2 to 3 days to generate a “bacterial film”.

### 3) Infection

(a) On the 5th day of germination (that is, after 3 days of dark culture and 2 days of light culture), take out the soybeans and cut them obliquely at 450 at the “green-white junction” of the hypocotyl.

(b) Use the soybean hypocotyl incision area to dip the engineered bacteria on the bacterial plate of engineered bacteria; put the infected soybeans into a nitrogen-free solid medium and culture in the dark for 2 days.

(c) Transfer the plants to a square dish with nitrogen medium and culture the hypocotyl in the dark for 6 days.

### 4) Hairy Root Culture

Take out the plant, remove the roots growing from the callus on the cut surface, and then place the plant in a nitrogen-containing liquid medium and culture for 9 to 10 days.

### 5) Identification and Screening of Transgenic Positive Roots

(a) Take out the plant, absorb the water on its roots with absorbent paper, and detect the positive roots under a stereofluorescence microscope, identify the ones with GFP green fluorescence as positive roots, keep only the strongest single male root and cut off the others, continue to culture the plant with one single positive root in in nitrogen-containing FM medium for 4-5 days, then move the plant to soil for culture and inoculation.

### (3) Identification and Phenotypic Analysis of GmNAS1 and GmNAP1 Expression Levels in Hairy Roots of Transgenic Plants

Twenty-five days after inoculation of Rhizobium USDA110, the nodule number, nodule weight and nodule nitrogenase activity of the hairy roots of the transgenic plants are measured. At the same time, the Real Time qRT-PCR method is used to analyze the gene expression of GmNAS1 and GmNAP1 in the nodules.

GmNAS1-qF:
CTGCCGTCAGCTATGCTAGG;
GmNAS1-qR:
CCAGAACGGAGACGCAATGTA;
GmNAP1-qF:
CATCCCCGACGGAACCAC;
GmNAP1-qR:
GCCCCTCTGTGACGACTCTA.

Utilize the successfully transformed Agrobacterium rhizogenes K599 to infect the soybean root callus to obtain soybean hairy roots. Twenty-five days after the soybean hairy roots are inoculated with Bradyrhizobium USDA110, the phenotypic statistics of the root nodules on the Ri-GmNAS1/NAP1 transgenic hairy roots are analyzed. It is found that the down-regulation of GmNAS1 and GmNAP1 expression does not affect the number and weight of root nodules, but significantly reduces the nitrogen-fixing ability of root nodules (FIG. 11).

### INDUSTRIAL APPLICABILITY

Construction and application of GmNAS1 and GmNAP1 gene editing vectors:

### (1) Construction of Plant Expression Vector CR-GmNAS1/NAP1

First, select 5′-CGGTGTCGAAAGTAATGACG-3′ and 5′-CTACTAGAGTCGTCGCCGAG-3′ as the sgRNA target sequences of GmNAS1 and GmNAP1, and connect the amplified sgRNA fragments into pBluescript-GmU6-tRNA to obtain the tRNA-gRNA intermediate vector; then digest GmU6-tRNA-gRNA fragment of the intermediate vector with PstI and KpnI, and ligate into the pCambia1300-35S-Cas9 vector to obtain the plant expression vector CR-GmNAS1/NAP1, which is then transferred into the W82 plant.

Wherein the pBluescript-GmU6-tRNA vector is obtained by: connecting the promoter sequence of soybean gene U6 (as shown in SEQ ID NO. 10) to the tRNA sequence (as shown in SEQ ID NO. 11), then inserting them together into the commercial vector pBluescript to obtain the pBluescript-GmU6-tRNA vector.

The primers are as follows:

CR-GmNAS1/NAP1-F:
ttcccggctggtgcacggtgtcgaaagt
aatgacggttttagagctagaaatagc;
CR-GmNAS1/NAP1-R:
gctatttctagctctaaaacctcggcga
cgactctagtagtgcaccagccgggaa.

### (2) Genetic Transformation of Soybean

In this study, the soybean cotyledon node transformation method mediated by Agrobacterium EHA105 is used for soybean transformation, which is mainly based on the transformation method reported by previous people (Luth et al 2015), and includes improvements on this basis.

### 1) Sterilization and Germination Soybean Seed

(a) Seed Sterilization. Pick the soybeans without damage and lesions and sterilize them with chlorine gas (measure 100 ml of sodium hypochlorite and place it in a 250 ml beaker, slowly add 5 ml of concentrated hydrochloric acid along the wall of the beaker, then seal and sterilize for 16 hours).

(b) Seed Germination. Place sterilized soybean seeds in a germination medium and incubate in the dark inside a 22° C. incubator for 16-24 hours to germinate.

### 2) Activation of Agrobacterium and Preparation of Infection Solution

Take 250 μl of frozen bacterial solution and spread on a LB plate with the corresponding antibiotics, and culture at 28° C. overnight. Use a disposable inoculation loop to scrape the bacterial film, hang in a liquid co-culture medium, and use a spectrophotometer to measure the concentration of the bacterial solution to make the final concentration OD₆₀₀=0.5˜0.6.

### 3) Explant Preparation and Infection

Keep the hypocotyl which is 3-5 mm long, separate the two cotyledons, remove the seed coat, and cut off the primary buds. Use a blade to make several cuts at the cotyledon node to obtain the cotyledon node explant for transformation. Place it in the solution for infection, and shake it on a horizontal rotator (rotational speed 50-80 r/min) for infection for 30 minutes.

### 4) Co-Culture

Pour away the bacterial solution for infection, transfer the explants to a solid co-culture medium covered with a layer of sterile filter paper and having 15 to 20 pieces per dish, and cultivate in the dark in a 22° C. incubator for 3-5 days.

### 5) Culture Screening and Plant Regeneration

(a) Bud induction culture: after co-culture for 3-5 days, transfer the explants to the bud induction medium, place 5 explants in each dish, and cultivate under the conditions of 16/8 hours photoperiod (light/dark) and 25° C., subculture once every 2 weeks, and subculture 2 times.

(b) Bud elongation culture: remove dead buds, cut off the cotyledons, transfer the explants to the bud elongation medium, place 5 explants in each dish, and cultivate under the conditions of 16/8 hours photoperiod (light/dark) and 25° C., subculture once every 3 weeks, and subculture 2-4 times.

(c) Root induction: when the elongated seedlings grow to 3 cm long, cut them out and transfer them to root induction medium, and cultivate under the conditions of 16/8 hours photoperiod (light/dark) and 25° C.

### 6) Seedling Hardening and Transplanting Culture

(a) When the regenerated plant takes root and grows two or more compound leaves, take out the plant, wash the culture medium at the root, plant it in a small flowerpot filled with sterilized vermiculite, and cultivate seedlings in an artificial intelligence incubator (temperature 25° C., photoperiod 16/8 hours, relative humidity 85% RH, light intensity 90 M/m²/s) for 5-7 days.

(b) After the seedlings are strong, transplant them into a large flower pot (nutrient soil: vermiculite=1:1) and move to a culture room (temperature 28±2° C., photoperiod 13.5/10.5 hours, relative humidity 40%-60% RH, light intensity 90 M/m²/s) and grow to maturity.

### (3) Identification and Phenotypic Analysis of CR-GmNAS1/NAP1 Editing Sites in Transgenic Plants

Design editing site identification primers based on the target sequences of sgRNA on GmNAS1 and GmNAP1, amplify the corresponding fragments and send them for sequencing. The sequencing results are analyzed and four function loss mutants of cr-nas1, cr-nap1, cr-nas1nap1-1 and cr-nas1nap1-2 are obtained. The nodule number, nodule weight and nodule nitrogenase activity on the 25th day after the mutant plants are inoculated with Rhizobia USDA110 are measured.

The primers are as follows:

GmNAS1-JD-F:
CATTCACATTCAGAAGAATGAGCAC;
GmNAS1-JD-R:
CTAGACGTGTTTTTTTTAGACACCG;
GmNAP1-JD-F:
TCATTCATCGAAACACAGAGAGAAG;
GmNAP1-JD-R:
TTGAAAAATGCTTTCCTCCAAACAG.

By utilizing gene editing technology, we obtain the functional knockout mutant cr-nas1 of GmNAS1, the functional knockout mutant cr-nap1 of GmNAP1, and the double knockout mutants cr-nas1nap1-1 and cr-nas1nap1-2 of GmNAS1 and GmNAP1. The root nodules of these four soybean mutants are phenotypically determined 25 days after inoculation with USDA110. It is found that under normal growth conditions, there is no significant difference in the number, weight and nitrogenase activity of these mutants compared with wild-type W82 plants. However, after three days of treatment with 3% (w/v) sucrose solution, we discover that the nitrogenase activity of wild-type plants is significantly higher than that of plants without sucrose, but the nitrogenase activities of cr-nas1, cr-nap1, cr-nas1nap1-1 and cr-nas1nap1-2 mutants are similar to those of plants without sucrose, which shows that GmNAS1 and GmNAP1 control the improvement of nitrogen fixation ability of soybean root nodules by sucrose (FIG. 12).

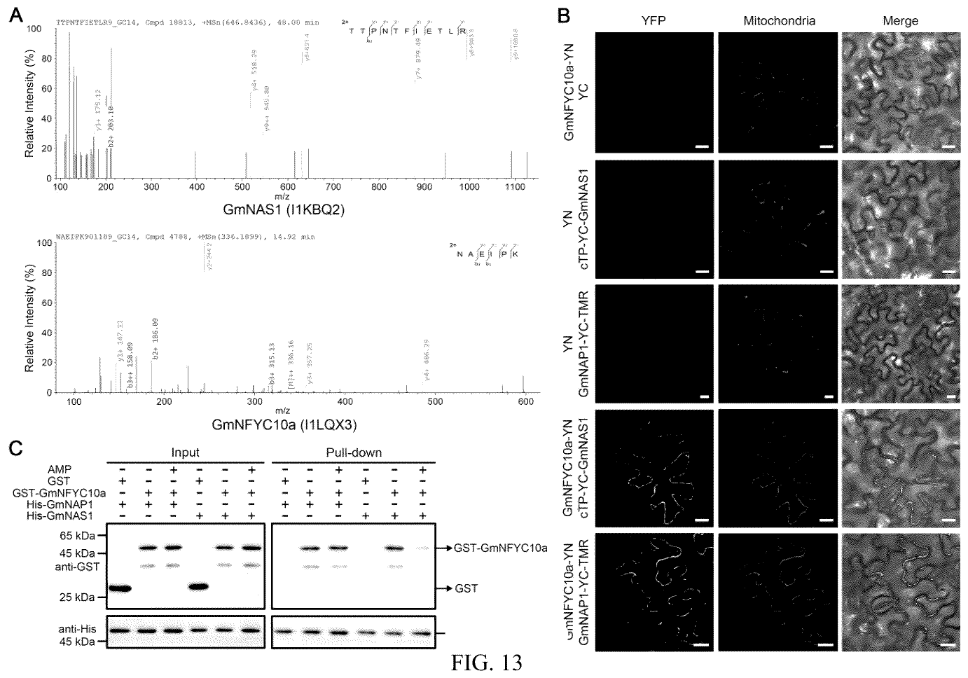

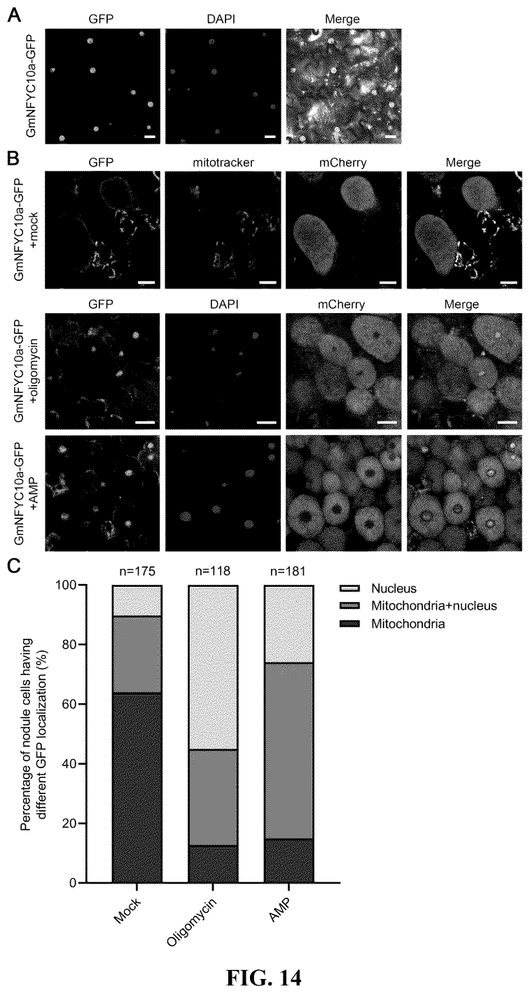

This application also uses co-immunoprecipitation coupled mass spectrometry analysis technology to search for candidate proteins that bind to GmNAP1. In the mass spectrometry results, a transcription factor GmNFYC10a is discovered. The pull-down method proves that GmNFYC10a can have interaction with GmNAS1/GmNAP1, and finds that the binding of AMP and GmNAS1 will significantly inhibit the interaction between GmNAS1 and GmNFYC10a. Bimolecular fluorescence complementation (bimolecular fluorescence complementation, BiFC) technology further demonstrates that GmNFYC10a and GmNAS1/GmNAP1 have interaction on mitochondria (FIG. 13). After GmNFYC10a-GFP fusion protein is injected into Nicotiana tabacum, we find that GmNFYC10a is localized in the nucleus, indicating that GmNFYC10a may be localized in mitochondria by having interaction with GmNAS1/GmNAP1, and the concentration of AMP in cells may affect the nuclear localization of GmNFYC10a. In order to verify this speculation, we first obtains GmNFYC10a-GFP transgenic hairy roots, and carrying out vibration sectioning of mature root nodules 25 days after inoculation with Rhizobia. It is found that the fluorescence is mainly localized in mitochondria, and after treating the sections with oligomycin or AMP for 2 hours, the fluorescence in the nucleus increased significantly, indicating that the increase in AMP content caused by the reduced energy status of root nodule cells will increase the content of GmNFYC10a in the nucleus by weakening the interaction between GmNFYC10a and GmNAS1 in mitochondria. (FIG. 14).

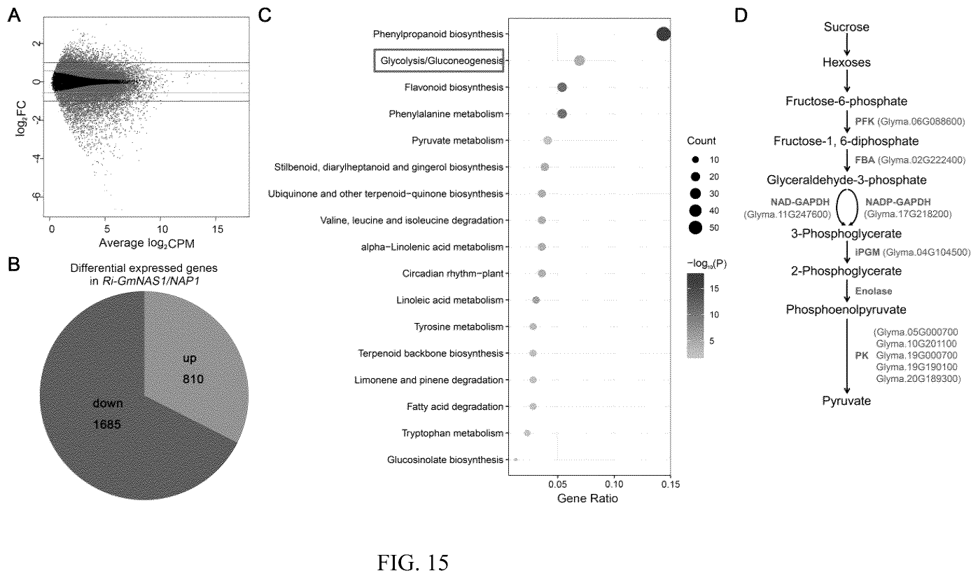

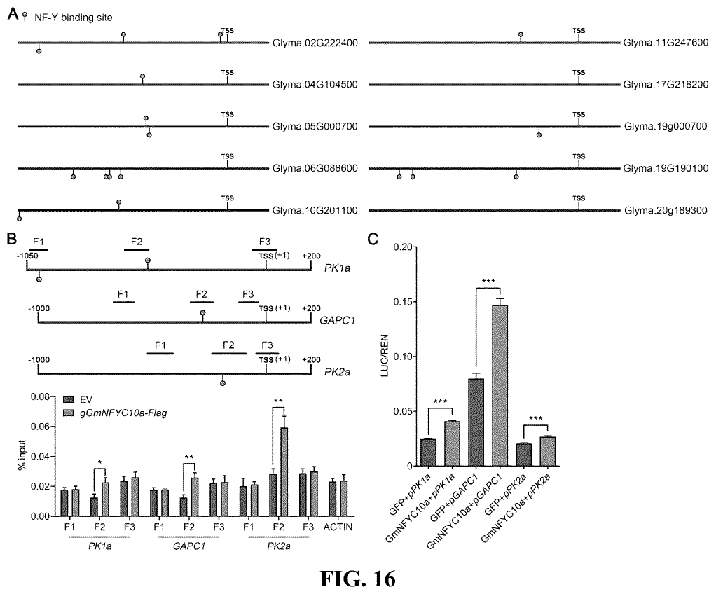

Transcriptome sequencing analysis is carried out on Nodules on Ri-GmNAS1/NAP1 transgenic hairy roots. We found that downregulation of GmNAS1/GmNAP1 expression affects many biological processes, including glycolysis and gluconeogenesis pathways, wherein the expression of 10 genes in the glycolysis pathway is significantly down-regulated, and these 10 genes included 5 pyruvate kinase genes (FIG. 15). Analysis of the promoters of these 10 glycolysis genes identified a binding motif of the nuclear factor Y (Nuclear Factor Y, NF-Y) transcription complex, indicating that the subunit GmNFYC10a of the NF-Y complex may regulate the expression of these glycolysis genes. The results of chromatin immunoprecipitation coupled quantitative PCR (chromatin immunoprecipitation qPCR, ChIP-qPCR) shows that GmNFYC10a does bind to the promoter region of the PK1a/GAPC1/PK2a gene. Further dual-luciferase activation assays (dual-luciferase assays, dual-LUC) demonstrates that GmNFYC10a can indeed activate the expression of glycolysis genes PK1a/GAPC1/PK2a (FIG. 16).

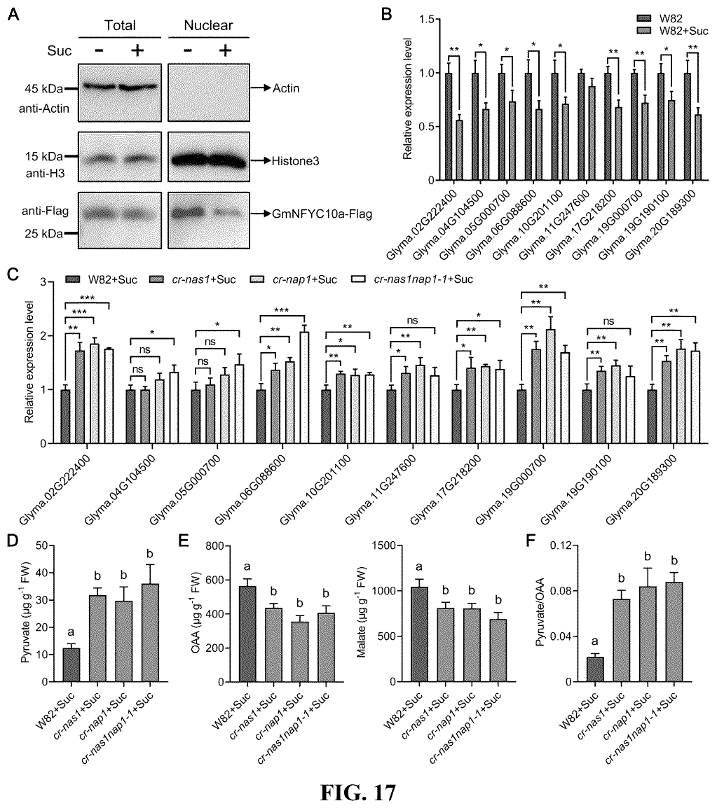

Previous experimental results show that the decrease in nodule energy status will increase the nuclear content of GmNFYC10a, while sucrose treatment can increase the nodule energy status, therefore we isolate the nuclei of soybean root nodule cells before and after sucrose treatment, and detect the protein level of GmNFYC10a in them. It is found that the protein content of GmNFYC10a in the nucleus decreases significantly after sucrose treatment, and the expression of glycolysis-related genes is also significantly down-regulated, while the expression of glycolysis-related genes in GmNAS1/GmNAP1 mutants cr-nas1, cr-nap1 and cr-nas1nap1-1 after sucrose treatment is significantly higher than that in wild-type W82, indicating that the decrease in GmNFYC10a nuclear content regulated by GmNAS1/GmNAP1 controls the inhibition of glycolytic gene expression under sucrose treatment (FIG. 17). Since half of these regulated glycolysis genes encode pyruvate kinase (catalyzing the conversion of PEP to pyruvate), we examine the pyruvate content in root nodules of W82, cr-nas1, cr-nap1 and cr-nas1nap1-1 under sucrose treatment. It is found that the pyruvate content in root nodules of cr-nas1, cr-nap1 and cr-nas1nap1-1 is significantly higher than that of the wild type, while the contents of oxaloacetate and malic acid converted from PEP are significantly lower than those of the wild type, indicating that the loss of function of GmNAS1 and GmNAP1 affects the distribution of PEP in soybean root nodules (FIG. 17).

The above descriptions are merely the preferred embodiments of the present invention and are not intended to be limiting. Any modifications, equivalent substitutions, improvements, etc. made within the spirit and principles of the present invention shall be included within the scope of protection of the present invention.

1: A protein that controls nitrogen fixation efficiency of soybean symbiosis, characterized in that: the protein is GmNAS1 protein and GmNAP1 protein, wherein an amino acid sequence of the GmNAS1 protein is shown in SEQ ID NO.4, and an amino acid sequence of GmNAP1 protein is shown in SEQ ID NO.5

2: The protein that controls nitrogen fixation efficiency of soybean symbiosis according to claim 1, characterized in that, the protein is localized in a mitochondrion and interacts with a transcription factor GmNFYC10a.

3: The protein that controls nitrogen fixation efficiency of soybean symbiosis according to claim 2, characterized in that, the protein localizes to the mitochondrion through a transmembrane domain at a carboxyl terminus.

4: The protein that controls nitrogen fixation efficiency of soybean symbiosis according to claim 2, characterized in that, the amino acid sequence of the transcription factor GmNFYC10a is shown in SEQ ID NO. 6.

5: A gene encoding the protein according to claim 4, characterized in that, a nucleotide sequence of a GmNAS1 gene encoding GmNAS1 protein is shown in SEQ ID NO.1; a nucleotide sequence of a GmNAP1 gene encoding GmNAP1 protein is shown in SEQ ID NO.2; a nucleotide sequence encoding the transcription factor GmNFYC10a is shown in SEQ ID NO. 3.

6: An overexpression vector containing the GmNAS1 gene and/or GmNAP1 gene according to claim 5.

7: An application of the protein according to claim 4 in improving plant symbiotic nitrogen fixation efficiency, enhancing plant environmental adaptability and increasing plant yields.

8: An application of the overexpression vector according to claim 6 in improving plant symbiotic nitrogen fixation efficiency, enhancing plant environmental adaptability, and increasing plant yield.

9: The application according to claim 8, characterized in that, comprising the steps of: transferring the overexpression vector containing the GmNAS1 gene and/or GmNAP1 gene through a genetic transformation method into a plant subject for improvement, and then screening and culturing the plant identified as transgenic positive to complete the application of enhancing the symbiotic nitrogen-fixing ability of soybean,
wherein for the GmNAS1 gene and/or GmNAP1 gene, a nucleotide sequence of a GmNAS1 gene encoding GmNAS1 protein is shown in SEQ ID NO.1; a nucleotide sequence of a GmNAP1 gene encoding GmNAP1 protein is shown in SEQ ID NO.2; a nucleotide sequence encoding the transcription factor GmNFYC10a is shown in SEQ ID NO. 3;
the GmNAS1 gene and the GmNAP1 gene encodes a GmNAS1 protein and a GmNAP1 protein respectively, and the GmNAS1 protein, the GmNAP1 protein or a combination of the GmNAS1 protein and the GmNAP1 protein is capable of controlling a nitrogen fixation efficiency of soybean symbiosis,
an amino acid sequence of a GmNAS1 protein is shown in SEQ ID NO.4, and an amino acid sequence of GmNAP1 Protein is shown in SEQ ID NO.5,
the GmNAS1 protein and the GmNAP1 protein are localized in a mitochondrion and interacts with a transcription factor GmNFYC10a, and an amino acid sequence of the transcription factor GmNFYC10a is shown in SEQ ID NO. 6.

10: The application according to claim 9, characterized in that, the improved plant is a leguminous plant.

11: The protein that controls nitrogen fixation efficiency of soybean symbiosis according to claim 3, characterized in that, the amino acid sequence of the transcription factor GmNFYC10a is shown in SEQ ID NO. 6.

12: A gene encoding the protein according to claim 11, characterized in that, a nucleotide sequence of a GmNAS1 gene encoding GmNAS1 protein is shown in SEQ ID NO.1; a nucleotide sequence of a GmNAP1 gene encoding GmNAP1 protein is shown in SEQ ID NO.2; a nucleotide sequence encoding the transcription factor GmNFYC10a is shown in SEQ ID NO. 3.

13: An overexpression vector containing the GmNAS1 gene and/or GmNAP1 gene according to claim 12.

14: An application of the gene according to claim 12 in improving plant symbiotic nitrogen fixation efficiency, enhancing plant environmental adaptability and increasing plant yields.

15: An application of the overexpression vector according to claim 13 in improving plant symbiotic nitrogen fixation efficiency, enhancing plant environmental adaptability, and increasing plant yield.

16: The application according to claim 15, characterized in that, comprising the steps of: transferring the overexpression vector containing the GmNAS1 gene, the GmNAP1 gene or a combination of the GmNAS1 gene and the GmNAP1 gene through a genetic transformation method into a plant subject for improvement, and then screening and culturing the plant identified as transgenic positive to complete the application of enhancing the symbiotic nitrogen-fixing ability of soybean,
wherein a nucleotide sequence of a GmNAS1 gene encoding GmNAS1 protein is shown in SEQ ID NO.1; a nucleotide sequence of a GmNAP1 gene encoding GmNAP1 protein is shown in SEQ ID NO.2; a nucleotide sequence encoding the transcription factor GmNFYC10a is shown in SEQ ID NO.1 SEQ ID NO. 3;
the GmNAS1 gene and the GmNAP1 gene encodes a GmNAS1 protein and a GmNAP1 protein respectively, and the GmNAS1 protein, the GmNAP1 protein or a combination of the GmNAS1 protein and the GmNAP1 protein is capable of controlling a nitrogen fixation efficiency of soybean symbiosis,
an amino acid sequence of a GmNAS1 protein is shown in SEQ ID NO.4, and an amino acid sequence of GmNAP1 protein is shown in SEQ ID NO.5, the GmNAS1 protein and the GmNAP1 protein are localized in a mitochondrion and interacts with a transcription factor GmNFYC10a, and an amino acid sequence of the transcription factor GmNFYC10a is shown in SEQ ID NO. 6.

17: The application according to claim 16, characterized in that, the improved plant is a leguminous plant.
